(19) United States
(12) Patent Application Publication
(10) Pub. No.: US 2013/0184492 A1
(43) Pub. Date: Jul. 18, 2013

(54) PROCESSES FOR PRODUCING HOOC-R-COOH COMPOUND ACIDS FROM FERMENTATION BROTHS CONTAINING NH4+ -OOC-R-COO-NH4+ COMPOUNDS

(71) Applicants: Olan S. Fruchey, Hurricane, WV (US); Leo E. Manzer, Wilmington (DE); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(72) Inventors: Olan S. Fruchey, Hurricane, WV (US); Leo E. Manzer, Wilmington (DE); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(21) Appl. No.: 13/695,308

(22) PCT Filed: Apr. 15, 2011
(86) PCT No.: PCT/US2011/032625

§ 371 Date: Dec. 10, 2012

Related U.S. Application Data
(60) Provisional application No. 61/329,940, filed on Apr. 30, 2010.

(51) Int. Cl. C07C 51/46

(52) U.S. Cl. CPC C07C 51/46
USPC 562/593

(57) ABSTRACT

A process for making HOOC—R—COOH compound acid from a clarified NH₄⁺⁻OOC—R—COO⁻NH₄⁺ compound-containing fermentation broth including distilling the broth under super atmospheric pressure at a temperature of >I00° C. to about 250° C. to form an overhead that comprises water and ammonia, and a liquid bottoms that Includes HOOC—R—COOH compound acid, and at least about 20 wt %. water; cooling the. bottoms to a temperature sufficient to cause the bottoms to separate into a liquid portion in contact with a solid portion that is substantially pure HOOC—R—COOH compound acid; separating the solid portion from the liquid portion; and recovering the solid portion.

### RELATED APPLICATIONS

This application claims the benefit of U.S. Provisional Application No. 61/329,940 filed Apr. 30, 2010, the subject matter of which is hereby incorporated by reference.

### TECHNICAL FIELD

This disclosure relates to processes for the direct production of HOOC—R—COOH compound acids from fermentation broths containing NH₄⁺⁻OOC—R—COO⁻NH₄⁺ compound.

### BACKGROUND

Certain carbonaceous products of sugar fermentation are seen as replacements for petroleum-derived materials for use as feedstocks for the manufacture of carbon-containing chemicals. One such product is HOOC—R—COOH compound.

It would therefore be desirable to have a process for the direct production of substantially pure HOOC—R—COOH compound acid from a NH₄⁺⁻OOC—R—COO⁻NH₄⁺ compound-containing fermentation broth.

### SUMMARY

We provide a process for making HOOC—R—COOH from a clarified NH₄⁺⁻OOC—R—COO⁻NH₄⁺-containing fermentation broth including distilling the broth under super atmospheric pressure at a temperature of >100° C. to about 300° C. to form an overhead that comprises water and ammonia, and a liquid bottoms that comprises HOOC—R—COOH and at least about 20 wt % water, cooling and/or evaporating the bottoms to attain a temperature and composition sufficient to cause the bottoms to separate into a liquid portion and a solid portion that is substantially pure HOOC—R—COOH, and separating the solid portion from the liquid portion.

We also provide a process for making HOOC—R—COOH from a clarified NH₄⁺⁻OOC—R—COO⁻NH₄⁺-containing fermentation broth including adding an ammonia separating solvent and/or a water azeotroping solvent to the broth, distilling the broth at a temperature and pressure sufficient to form an overhead that comprises water and ammonia, and a liquid bottoms that comprises HOOC—R—COOH and at least about 20 wt % water, cooling and/or evaporating the bottoms to attain a temperature and composition sufficient to cause the bottoms to separate into a liquid portion and a solid portion that is substantially pure HOOC—R—COOH, and separating the solid portion from the liquid portion.

We further provide a process for making HOOC—R—COOH from a clarified NH₄⁺⁻OOC—R—COOH-containing fermentation broth including distilling the broth under super atmospheric pressure at a temperature of >100° C. to about 300° C. to form an overhead that comprises water and ammonia, and a liquid bottoms that comprises HOOC—R—COOH and at least about 20 wt % water, cooling and/or evaporating the bottoms to attain a temperature and composition sufficient to cause the bottoms to separate into a liquid portion and a solid portion that is substantially pure HOOC—R—COOH, and separating the solid portion from the liquid portion.

We further provide a process for making HOOC—R—COOH from a clarified NH₄⁺⁻OOC—R—COOH-containing fermentation broth including adding an ammonia separating solvent and/or a water azeotroping solvent to the broth, distilling the broth at a temperature and pressure sufficient to form an overhead that includes water and ammonia, and a liquid bottoms that comprises HOOC—R—COOH, and at least about 20 wt % water, cooling and/or evaporating the bottoms to attain a temperature and composition sufficient to cause the bottoms to separate into a liquid portion and a solid portion that is substantially pure HOOC—R—COOH, and separating the solid portion from the liquid portion.

### BRIEF DESCRIPTION OF THE DRAWINGS

FIG. 1 is a block diagram of a process for the production of HOOC—R—COOH from a NH₄⁺⁻OOC—R—COO⁻NH₄⁺-containing fermentation broth.

### DETAILED DESCRIPTION

It will be appreciated that at least a portion of the following description is intended to refer to representative examples of processes selected for illustration in the drawings and is not intended to define or limit the disclosure, other than in the appended claims.

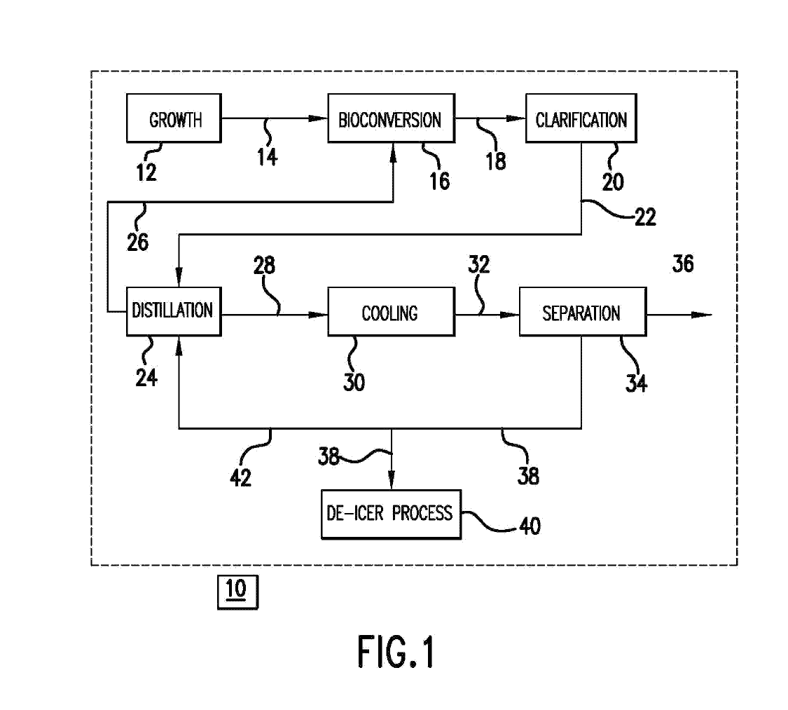

Our processes may be appreciated by reference to FIG. 1, which shows in block diagram form one representative example 10 of our methods.

HOOC—R—COOH compound acids, NH₄⁺⁻OOC—R—COOH compounds and NH₄⁺³¹ OOC—R—COO⁻NH₄⁺ compounds; wherein R may be but is not limited to of CH₂, CH═CH, CH₂—CH(OH), (CH₂)₃, C(CH₃)═CH, CH₂—C═CH₂, CH═CH—CH═CH, (CH₂)₈ and (CH₂)₁₀ are referred to herein. Exemplary, HOOC—R—COOH compound acids include but are not limited to malonic acid, fumaric acid, malic acid, glutaric acid, citraconic acid, itaconic acid, muconic acid, sebacic acid and dodecanedioic acid; wherein R is selected from the group consisting of CH₂, CH═CH, CH₂—CH(OH), (CH₂)₃, C(CH₃)═CH, CH₂—C═CH₂, CH═CH—CH═CH, (CH₂)₈ and (CH₂)₁₀. Exemplary, NH₄⁺⁻OOC—R—COOH compounds include but are not limited to monoammonium malonate, monoammonium fumarate, monoammonium malate, monoammonium glutarate, monoammonium citraconate, monoammonium itaconate, monoammonium muconate, monoammonium sebacate and monoammonium dodecanedionate; wherein R is selected from the group consisting of CH₂, CH₂—CH(OH), (CH₂)₃, C(CH₃)═CH, CH₂—C═CH₂, CH═CH—CH═CH, (CH₂)₈ and (CH₂)₁₀. Exemplary, NH₄⁺⁻OOC—R—COO⁻NH₄⁺ compounds include but are not limited to diammonium malonate, diammonium fumarate, diammonium malate, diammonium glutarate, diammonium citraconate, diammonium itaconate, diammonium muconate, diammonium sebacate and diammonium dodecanedionate; wherein R is selected from the group consisting of CH₂, CH═CH, CH₂—CH(OH), (CH₂)₃, C(CH₃)═CH, CH₂—C═CH₂, CH═CH—CH═CH, (CH₂)₈ and (CH₂)₁₀.

A growth vessel 12, typically an in-place steam sterilizable fermentor, may be used to grow a microbial culture (not shown) that is subsequently utilized for the production of the NH₄⁺⁻OOC—R—COO⁻NH₄⁺ compound-containing, NH₄⁺⁻OOC—R—COOH compound-containing and/or HOOC—R—COOH compound acid-containing fermentation broth. Such growth vessels are known in the art and are not further discussed.

The microbial culture may comprise microorganisms capable of producing NH₄⁺⁻OOC—R—COO⁻NH₄⁺ compounds/HOOC—R—COOH compound acids from fermentable carbon sources such as carbohydrate sugars (e.g., glucose), cyclohexanol, alkanes (e.g., n-alkanes), plant based oils and others. Representative examples of microorganisms include Escherichia coli (E. coli), Aspergillus niger, Aspergillus terreus, Aspergillus itaconicus, Corynebacterium glutamicum (also called Brevibacteriumflavum), Enterococcus faecalis, Veillonella parvula, Actinobacillus succinogenes, Paecilomyces varioti, Saccharomyces cerevisiae, Candida tropicalis, Bacteroides fragilis, Bacteroides ruminicola, Bacteroides amylophilus, Klebsiella pneumoniae, Phanerochaete chrysorporium, Corynebacterium nitrilophilus, Gordona terrae, Rhodococcus rhodochrous, Aspergillus flavus, Aspergillus parasiticus, Aspergillus oryzae, Rhodotorula mucilanginosa, Pseudomonas putida, Candida maltosa, mixtures thereof and the like. Additionally, other microbes such as Escherichia spp., Aspergillus spp., Ustilago spp., Corynebacterium spp. (also called Brevibacteriumflavum), Enterococcus spp., Veillonella spp., Actinobacillus spp., Paecilomyces spp., Saccharomyces spp., Candida spp., Bacteroides spp., Klebsiella spp., Phanerochaete spp., Gordona spp., Rhodococcus spp., Rhodotorula spp. and Pseudomonas spp. as well as Torulopsis spp., Debaryomyces spp., Hansenula spp., Pichia spp., Rhizopus species (spp.) such as Rhizopus nigricans, Rhizopus arrhizus, Rhizopus oryzae, Rhizopus oligosporus, Rhizopus microsporous, Rhizopus circinans, Rhizopus formosa; Mucor spp.; Cuninghamella spp.; Circinella spp.; Lactobacillus spp.; Escherichia coli may be used, as appropriate, in the preparation of fermentation broths containing NH₄⁺⁻OOC—R—COO⁻NH₄⁺ compounds.

Preferred microorganisms for fumaric acid production include Rhizopus oryzae Went et Prinsen Geerligs, teleomorph strain NRRL 1526 having ATCC accession number 10260 (also known as Rhizopus arrhizus NRRL 1526); Rhizopus oligosporus Saito, telemorph strain NRRL 2710 having ATCC accession number 22959; Rhizopus microsporus van Tieghem, telemorph deposited at Rhizopus cohnii Berlese et De Toni, teleomorph strain U-1 having ATCC accession number 46436; Rhizopus circinans van Tieghem, teleomorph strain NRRL 1474 having ATCC accession number 52315; Rhizopus oryzae Went et Prinsen Geerligs, teleomorph strain NRRL 2582 having ATCC accession number 52918; Rhizopus oryzae Went et Prinsen Geerligs, teleomorph strain NRRL 395 having ATCC accession number 9363; and Rhizopus oryzae Went et Prinsen Geerligs, teleomorph deposited as Rhizopus stolonifer (Ehrenberg: Fries) Lind, teleomorph strain designated Waksman 85 having ATCC accession number 13310. Other microorganisms suitable for fumaric acid production include Lactobacillus host strains lacking the malolactate enzyme, fumarase and fumarate dehydrogenase. Such microorganisms can produce FA from monosaccharides such as glucose, sucrose, fructose and xylose; disaccharides such as maltose; polysaccharides such as starches and other carbon sources such as molasses, invert high test molasses, syrups, grains, malted grains, cereal products, starch hydrolysate, corn steep liquor and the like. Additionally, these microorganisms can produce FA when cultured on solid mediums such as potato dextrose agar (PDA; ATCC medium 336) or its fluid medium equivalents.

Fermentation broths containing fumaric acid can be produced from the Rhizopus oryzae Went et Prinsen Geerligs, teleomorph strain NRRL 395 having ATCC accession number 9363, or other such strains, by culture in a sterilized liquid medium comprising 100 g/L glucose, 0.6 g/L urea, 0.5 ml/L corn steep liquor, 0.3 g/L KH₂PO₄, 0.4 g/L MgSO₄. 7H₂O; 0.044 g/L ZnSO₄. 7H₂O, 0.01 g/L ferric tartrate, 100 g/L CaCO₃; 300 μg/mL polyoxyethylene sorbitan monooleate (TWEEN 40™) and 300 μg/mL polyoxyethylene sorbitan monopalmitate (TWEEN 20™) in water for 4 days at 32° C. as described in U.S. Pat. No. 4,564,594 the subject matter of which is incorporated herein by reference.

Fermentation broths containing fumaric acid can be produced from Rhizopus oryzae strains by culture in a sterilized liquid medium prepared by adding 15 g invert sugar produced by enzymatic or acid inversion of high test molasses, 9.75 g calcium carbonate, 0.0375 g magnesium sulfate heptahydrate; 0.15 g potassium acid phosphate, 1.5 mg ferric sulfate and about 0.02% (w/w of the liquid fermentation culture medium) to about 0.25% (w/w of the liquid fermentation culture medium) of either ammonium sulfate or urea in 140 mL of water and sterilizing the medium. This sterilized liquid medium is then inoculated with Rhizopus oryzae and the fermentation may be conducted for 7 days at 28° C. to 32° C. with the addition of about 0.02% (w/w of the liquid fermentation culture medium) to about 0.12% (w/w of the liquid fermentation culture medium) of either ammonium sulfate or urea at 24 h, 48 h, 72 h and 96 h after inoculation, or at combinations of these times, as described in U.S. Pat. No. 2,912,363 the subject matter of which is incorporated herein by reference.

Preferred microorganisms for diammonium malonate/malonic acid production include Phanerochaete chrysorporium Burdsall, teleomorph strain VKM F-1767 having ATCC accession number 24725™; Corynebacterium nitrilophilus Akio et al. strain C42 having ATCC accession number 21419™; Gordona terrae strain MA-1 having National Institute of Bioscience and Human-Technology (Higashi 1-chome, Tsukuba-shi, Ibarakiken, Japan) accession number FERM-BP-4535; and Rhodococcus rhodochrous (Zopf) Tsukamura emend. Rainey et al. deposited as Nocardia lucida strain IMRU 3890 having ATCC accession number 33025™ which may produce malonic acid, or malonic acid monoesters represented by the formula HOOC—CH₂—COOR′ (where R′ is an alkenyl, aryl, aralkyl or C₃₋₂₀ alkyl), from monosaccharides such as glucose, polysaccharides such as starches and cellulose, and other carbon sources such as wood, corn and cyanoacetic acid esters represented by the formula NC—CH₂—COOR′ (where R′ is an alkenyl, aryl, aralkyl or C₃₋₂₀ alkyl). Additionally, these microorganisms may produce malonic acid when cultured on solid mediums such as potato dextrose agar (ATCC medium 336), yeast extract-glucose medium (ATCC medium 25), nutrient agar (ATCC medium 3) or fluid medium equivalents of these or malonic acid esters when cultured in such mediums with cyanoacetic acid esters.

Fermentation broths containing diammonium malonate/malonic acid may be produced from the Phanerochaete chrysorporium Burdsall, teleomorph strain VKM F-1767 having ATCC accession number 24725™, or other such strains, by culture in a liquid medium comprising a 1% carbon source of glucose, cellulose or wood as a liquid stationary culture at 37° C., as described by Abbas et al., 47 Curr. Genet. 49 (2005) the subject matter of which is incorporated herein by reference, that is flushed with water-saturated O₂ on day 2 after inoculation and every 3 days thereafter. The production of diammonium malonate/malonic acid by Phanerochaete chrysoporium is also described in National Laboratory Field Work Proposal Final Report having FWP/OTIS Number: DE-AC06-76RL0 1830 #41721 and by Kingsley, M T; Romine, R A; and Lasure, L L “Effects of Medium Composition on Morphology and Organic Acid Production in Phanerochaete chrysosporium” in a poster presentation at the Annual Meeting of the American Society of Microbiology the subject matter of which is incorporated herein by reference.

Fermentation broths containing malonic acid esters may be produced from Gordona terrae strain MA-1 having National Institute of Bioscience and Human-Technology (Higashi 1-chome, Tsukuba-shi, Ibarakiken, Japan) accession number FERM-BP-4535, Corynebacterium nitrilophilus Akio et al. strain C42 having ATCC accession number 21419™ or Rhodococcus rhodochrous (Zopf) Tsukamura emend. Rainey et al. deposited as Nocardia lucida strain IMRU 3890 having ATCC accession number 33025™ by inoculation of one of these microorganisms into 3 ml of sterilized LB medium (1% polypeptone, 0.5% yeast extract, 0.5% NaCl) and culture at 30° C. for 24 h with shaking. One ml of the resultant cell culture liquid may be inoculated into 100 ml of a sterilized medium A (pH 7.2) comprising glycerol (1.0%), isovaleronitrile (0.2%), yeast extract (0.02%), KH₂PO₄ (0.2%), MgSO₄. 7H₂O (0.02%), FeSO₄. 7H₂O (10 ppm), CoCl₂. 4H₂O (10 ppm), CaCl₂. 2H₂O (1 ppm) and MnCl₂. 4H₂O (7 ppm) and cultured at 30° C. for 48 hrs. After completion of the cultivation, the culture liquid is centrifuged. The total volume of the resultant cell pellet is then washed with deionized water and suspended in 100 ml of 50 mM phosphate buffer (pH 7.0). Ideally, the turbidity of the cell suspension is OD_{630 nm} ˜5.5-5.6. To this cell suspension 1.00 g of ethyl cyanoacetate is added as a substrate, when using the Gordona terrae strain or the Corynebacterium nitrilophilus strain, and reacted at 30° C. for 1 h to form a fermentation broth containing monoethyl malonate. When using the Rhodococcus rhodochrous strain, 1.00 g of n-propyl cyanoacetate is added as a substrate and reacted at 30° C. for 1 h to form a fermentation broth containing mono-n-propyl malonate. Analysis by high performance liquid chromatography (HPLC; column: TSKgel ODS-120A (Tosoh Corp.), 4.6 mm I.D.×25 cm; mobile phase: 5% acetonitrile, 95% water, 0.1% phosphoric acid; flow rate: 0.5 ml/min; detection: UV 220 nm) may be performed to confirm conversion of the ethyl cyanoacetate to monoethyl malonate or the conversion of n-propyl cyanoacetate to mono-n-propyl malonate.

After completion of the reaction, cells may be removed from the fermentation broth by centrifugation. 2N HCl may be added to the resultant solution to adjust the pH to 2.0. Thereafter, monoethyl malonate, the reaction product, may be extracted from the broth with ethyl acetate. Anhydrous sodium sulfate may be added to the resultant organic layer for dehydration, and the solvent can be removed by distillation to obtain monoethyl malonate was at a yield of about 89.9%. The production of such malonic acid esters having the formula HOOC—CH₂—COOR′ from cyanoacetic acid esters represented by the formula NC—CH₂—COOR′ by Gordona terrae strain MA-1 having National Institute of Bioscience and Human-Technology (Higashi 1-chome, Tsukuba-shi, Ibarakiken, Japan) accession number FERM-BP-4535 and Corynebacterium nitrilophilus Akio et al. strain C42 having ATCC accession number 21419™ is also described in U.S. Pat. No. 6,238,896 the subject matter of which is incorporated herein by reference.

Alternatively, after completion of the reaction at 30° C. for 1 h and the removal of cells, a saponification reaction may be performed by treating the fermentation broth from the Gordona terrae strain or the Corynebacterium nitrilophilus strains with a quantity of a strong base such as KOH sufficient to remove the monoethyl group from the monoethyl malonate ester and to form the alcohol CH₃—CH₂—OH (ethanol) and HOOC—CH₂—COO⁻. The resulting HOOC—CH₂—COO⁻ may then be treated to form the appropriate fermentation broth.

Similarly, after completion of the reaction at 30° C. for 1 h and the removal of cells, a saponification reaction may be performed by treating the fermentation broth from the Rhodococcus rhodochrous strain with a quantity of a strong base such as KOH sufficient to remove the mono-n-propyl group from the mono-n-propyl malonate ester and to form the alcohol CH₃—CH₂—CH₂—OH (n-propanol) and HOOC—CH₂—COO⁻ (which may be in the form of a salt). The resulting HOOC—CH₂—COO⁻ may then be treated to form the appropriate fermentation broth.

This approach of producing HOOC—CH₂—COOR′ esters in fermentation broths followed by de-esterification to form HOOC—CH₂—COO⁻ and R′—OH via saponification reactions, and the like to produce a fermentation broth can also be used with malonate esters produced from other cyanoacetic acid esters represented by the formula NC—CH₂—COOR′.

Preferred microorganisms for diammonium malate/malic acid production include Aspergillus flavus Link, anamorph strain A-114 having ATCC accession number 13697™; Aspergillus flavus Link, anamorph strain A-57 having ATCC accession number 13698™; Aspergillus parasiticus Speare, anamorph strain WB 465 having ATCC accession number 16869™; Aspergillus parasiticus Speare, anamorph strain A-237 having ATCC accession number 13696™; and Aspergillus oryzae (Ahlburg) Cohn, anamorph strain NRRL 3488 having ATCC accession number 56747™ which produce diammonium malate/malic acid from monosaccharides such as glucose, sucrose, fructose, galactose sorbose and xylose; disaccharides such as maltose; polysaccharides such as starches and other carbon sources such as sorbitol, glycerol, molasses, corn and the like. Additionally, these microorganisms may produce diammonium malate/malic acid when cultured on solid mediums such as Czapek's agar (ATCC medium 312) and potato dextrose agar (PDA; ATCC medium 336) or fluid medium equivalents of these.

Fermentation broths containing diammonium malate/malic acid may be produced from the Aspergillus flavus Link, anamorph strain A-114 having ATCC accession number 13697™, Aspergillus flavus Link, anamorph strain A-57 having ATCC accession number 13698™, Aspergillus parasiticus Speare, anamorph strain WB 465 having ATCC accession number 16869™, Aspergillus parasiticus Speare, anamorph strain A-237 having ATCC accession number 13696™ and Aspergillus oryzae (Ahlburg) Cohn, anamorph strain NRRL 3488 having ATCC accession number 56747™, or other such strains, by aerobic culture in 1 L of a liquid medium comprising 10% glucose, 0.6% peptone, 0.015% KH₂PO₄, 0.015% K₂HPO₄, 0.01% MgSO₄. 7H₂O, 0.01% CaCl₂. 2H₂O, 5 mg of NaCl, 5 mg of FeSO₄. 7H₂O and distilled water (all percentages here are by weight per volume, i.e. grams per cubic centimeter). The 1 L solution is then divided into 30 mL portions each of which is placed into a separate 250 ml flask and is sterilized by heating under pressure at 120° C. for 15 min. 4% of CaCO₃ (the percentage here is by weight per volume, i.e. grams per cubic centimeter), which was separately sterilized by dry heating, is then added to each of the flasks. Thereafter a microorganism such as Aspergillus flavus Link, anamorph strain A-114 having ATCC accession number 13697™, Aspergillus flavus Link, anamorph strain A-57 having ATCC accession number 13698™, Aspergillus parasiticus Speare, anamorph strain WB 465 having ATCC accession number 16869™, Aspergillus parasiticus Speare, anamorph strain A-237 having ATCC accession number 13696™ and Aspergillus oryzae (Ahlburg) Cohn, anamorph strain NRRL 3488 having ATCC accession number 56747™, or other such strains or combinations of these, may be inoculated into the culture solution. The microorganisms are then cultivation for 7 days at 28° C. on a rotary shaker operating at 200 rpm to produce a fermentation broth with a pH of about 5.4 to about 6.2 that contains malic acid (L-malic acid). Typically, the fermentation broths produced may contain from about 21.8 mg/mL to about 32.6 mg/mL of L-malic acid. The production of fermentation broths from Aspergillus flavus, Aspergillus parasiticus and Aspergillus oryzae in the medium described above, and other mediums such as solid equivalents of these, is also described in U.S. Pat. No. 3,063,910 the subject matter of which is incorporated herein by reference.

Preferred microorganisms for diammonium glutarate/glutaric acid production include Candida tropicalis (Castellani) Berkhout, anamorph strain OH23 having ATCC accession number 24887™, Rhodotorula mucilanginosa (Jorgensen) Harrison var. mucilaginosa, anamorph deposited as Rhodotorula rubra (Demme) Lodder, anamorph strain AKU 4817 having ATCC accession number 64041™ and the lysine-requiring Saccharomyces cerevisiae strain C-1 which produce diammonium glutarate/glutaric acid from monosaccharides such as glucose and other carbon sources such as azelaic acid, n-pentadecane, and the like. Additionally, these microorganisms can be cultured on mediums such as malt extract agar (Blakeslee's formula; ATCC medium 325); YM agar (ATCC medium 200) or YM broth (ATCC medium 200) or equivalents of these.

Fermentation broths containing diammonium glutarate/glutaric acid may be produced from the Candida tropicalis (Castellani) Berkhout, anamorph strain OH23 having ATCC accession number 24887™, or other such strains, by culture at 32° C. in a liquid medium containing 300 mg of NH₄H₂PO₄, 200 mg of KH₂PO₄, 100 mg of K₂HPO₄, 50 mg of MgSO₄. 7H₂O, 1 μg of biotin, 0.1% (w/v) yeast extract and about 1% (v/v) n-pentadecane in 100 ml of distilled water. The procedure for producing fermentation broths from media containing n-pentadecane by culturing Candida tropicalis (Castellani) Berkhout, anamorph strain OH23 having ATCC accession number 24887 is also described in Okuhura et al., 35 Agr. Biol. Chem. 1376 (1971) the subject matter of which is incorporated herein by reference.

Fermentation broths containing diammonium glutarate/glutaric acid can be produced from the Rhodotorula mucilanginosa (Jorgensen) Harrison var. mucilaginosa, anamorph deposited as Rhodotorula rubra (Demme) Lodder, anamorph strain AKU 4817 having ATCC accession number 64041™, or other such strains, as follows. The Rhodotorula mucilanginosa strain is cultured at 28° C. for 38 h with shaking in a 500 ml flask containing 100 mL of a liquid medium comprising 1.0% glucose, 0.5% peptone, 0.3% malt extract and 0.3% yeast extract (all percentages here are by weight per volume, i.e. grams per cubic centimeter). The initial pH is then adjusted to 6.0. After growth, cells are harvested and washed three times with saline solution and then suspended in M/200 potassium phosphate buffer (pH 7.5). The cell suspension (about 20-40 mg/mL) is then incubated with azelaic acid (4 mg/mL) in 3 ml of 0.2 M Tris-HCl buffer (pH 7.5) at 28° C. for 24 h with shaking Cells are then removed from this fermentation broth by centrifugation to produce a clarified fermentation broth containing glutaric acid. The fermentation broth. The production of fermentation broths from Rhodotorula mucilanginosa strains according to the process described above is also described by Ohsugi et al., 48 Agric. Biol. Chem. 1881 (1984) the subject matter of which is incorporated herein by reference.

Fermentation broths containing diammonium glutarate/glutaric acid may be produced from the lysine-requiring Saccharomyces cerevisiae strain C-1 as follows. A sterilized medium having a pH of 5.5 and comprising 75 g glucose, 7.5 g NH₄H₂PO₄, 3 g KH₂PO₄, 0.183 mg MgSO₄. 7H₂O, 1.13 mg nicotinamide, 0.15 g biotin, 3.8 mg calcium pantothenate, 75 mg i-inositol, 33 mg thiamine hydrochloride, 9 mg pyridoxine hydrochloride, 13.2 mg ZnSO₄. 7H₂O, 7.88 mg FeSO₄(NH₄)₂SO₄.6H₂O, 0.73 mg CuSO₄. 5H₂O and 100 mg L-lysine.HCl in 1 L of water is prepared. The medium is then distributed in 200 ml portions into 500 ml Erlenmeyer flasks which are inoculated with the lysine-requiring Saccharomyces cerevisiae strain C-1. The lysine-requiring Saccharomyces cerevisiae strain C-1 is described by Mattoon et al., 51 Biochim. Et Biophys. Acta 615 (1961), the subject matter of which is incorporated herein by reference and was derived from the Saccharomyces cerevisiae C strain. Fermentations may then be performed at 30° C. on a rotary shaker for 9 days after which cells are removed from the fermentation broth by centrifugation. The resulting fermentation broth may be reduced 10 fold under reduced pressure at 35° C. The production of fermentation broths from the lysine-requiring Saccharomyces cerevisiae strain C-1 according to the process described above is also described by Matton and Haight, 237 J. Biol. Chem. 3486 (1962) the subject matter of which is incorporated herein by reference.

Preferred microorganisms for diammonium itaconate/itaconic acid production include Aspergillus terreus Thom, anamorph strain NRRL 1960 having ATCC accession number 10020™; Aspergillus terreus strain RC4′ derived from the NRRL 1960 strain; Aspergillus terreus strain CM85J derived from the NRRL 1960 strain; Aspergillus terreus Thom, anamorph strain NRRL 265 having ATCC accession number 10029™; Aspergillus terreus Thom, anamorph strain MF4845 having ATCC accession number 20542™; Aspergillus terreus Thom, anamorph strain K 26 having ATCC accession number 32359™; Aspergillus terreus Thom, anamorph strain 14/II having ATCC accession number 32587™; Aspergillus terreus Thom, anamorph strain 21/I having ATCC accession number 32588™; Aspergillus terreus Thom, anamorph strain 25/III having ATCC accession number 32589™; Aspergillus terreus Thom, anamorph strain K having ATCC accession number 32590™; Aspergillus terreus Thom, anamorph strain 3 having ATCC accession number 36364™; Aspergillus terreus TN484-M1; Aspergillus itaconicus Kinshita, anamorph strain NRRL 161 having ATCC accession number 56806™; and Basidiomycetes of the genus Ustilago which produce diammonium itaconate/itaconic acid from monosaccharides such as glucose, sucrose, fructose, xylose and arabinose; disaccharides such as lactose; polysaccharides such as starches and other carbon sources such as molasses, sago starch, sago starch hydrolysate, corn steep liquor and the like. Additionally, these microorganisms may produce diammonium itaconate/itaconic acid when cultured on solid mediums such as Czapek's agar (ATCC medium 312), potato dextrose agar (PDA; ATCC medium 336), malt agar medium (ATCC medium 323), malt extract agar (Blakeslee's formula; ATCC medium 325); malt extract agar (ATCC medium 324) potato dextrose yeast agar (PDY; ATCC medium 337) or fluid medium equivalents of these.

Fermentation broths containing diammonium itaconate/itaconic acid may be produced from the Aspergillus terreus strain TN484 strain, or other such strains, by culture in a liquid medium comprising 140 g/L sago starch hydrolysate, 1.8 g/L corn steep liquor, 1.2 g/L MgSO₄. 7H₂O and 2.9 g/L NH₄NO₃ in water as described by Dwiarti et al., 98 Bioresour. Technol. 3329 (2007) the subject matter of which is incorporated herein by reference.

Fermentation broths containing diammonium itaconate/itaconic acid may be produced from the Aspergillus terreus strain RC4′ derived from the NRRL 1960 strain and Aspergillus terreus strain CM85J derived from the NRRL 1960 strain by culture at 35° C. in a liquid medium comprising glucose as described in French patent 8805487 and by Gillet et al., 177 J. Bact. 3573 (1995) the subject matter of which is incorporated herein by reference.

Fermentation broths containing diammonium itaconate/itaconic acid may be produced from the Aspergillus terreus Thom, anamorph strain MF4845 having ATCC accession number 20542™ by culture in a liquid, lactose based medium (LBM) or an identical medium containing glucose instead of lactose as described by Lai et al., 104 J. Biosci. Bioeng. 9 (2007) the subject matter of which is incorporated herein by reference.

Fermentation broths containing diammonium itaconate/itaconic acid may be produced from the Aspergillus terreus Thom, anamorph strain NRRL 1960 having ATCC accession number 10020™ by culture in an aqueous medium. This aqueous medium may be prepared by making an aqueous solution of wheat starch having 350 g/L of dry solids by homogenization, adjusting the pH to 6.5 and adding 0.175 g/L of the liquefying enzyme (TERMAMYL 120L® marketed by Novozymes A/S, Denmark). This aqueous solution may then be introduced into a steam injection sterilizer. The temperature may then be maintained at 100° C. to 105° C. for 7 min, cooled to 95° C. and may be maintained at this temperature for 2 hours in an agitated tank and then cooled to 35° C. to produce a fluidized starch solution. Next, a quantity of the fluidized starch solution containing 130 kg of dry solids may be introduced into a 1,400 L fermenter. The following materials may then be added to this fermenter:

(i) a nutritive solution sterilized for 30 min at 100° C., containing 0.5 kg corn extract, 3.45 kg magnesium chloride, 0.3 kg magnesium sulfate, 0.9 kg urea, 0.4 kg sodium chloride, 0.033 kg zinc sulfate, 0.05 kg monopotassium phosphate, 1 kg calcium chloride, 0.06 kg copper sulfate and 0.3 kg sulfuric acid (the pH was 3.6); and

(ii) 0.29 kg amyloglucosidase (AMG 200L®, marketed by Novo Industry marketed by Novozymes A/S, Denmark).

This production medium, the final volume of which may be adjusted to 1,000 L with sterile water, may then be agitated, aerated and inoculated with 20 L of Aspergillus terreus Thom, anamorph strain NRRL 1960 having ATCC accession number 10020™ culture, aged 35 hours that may be previously prepared at 32 to 35° C. in a fermenter containing 25 g/L glucose, 4.5 g/L magnesium sulfate, 0.4 g/L sodium chloride, 0.004 g/L zinc sulfate, 0.L g/L monopotassium phosphate, 0.5 g/L corn extract, 2.0 ammonium nitrate and 0.5 g/L sulfuric acid. The temperature of the medium in the inoculated 1,400 L fermenter may then be maintained at 32° C. to 35° C. and the fermentation may be terminated after the sugar was consumed and when the acidity is maximal and stable. Wort samples may be periodically withdrawn from the 1,400 L fermenter to evaluate the itaconic acid content. This method of diammonium itaconate/itaconic acid production from Aspergillus terreus Thom, anamorph strain NRRL 1960 having ATCC accession number 10020™ culture and similar methods for itaconic acid production are also described in U.S. Pat. No. 5,231,016 the subject matter of which is incorporated herein by reference.

Fermentation broths containing diammonium itaconate/itaconic acid may be produced from the Aspergillus terreus Thom, anamorph strain NRRL 1960 having ATCC accession number 10020™ by culture at 32° C. to 35° C. in an aqueous medium comprising between 0 g to 110 g of sucrose and 0 g to 100 g of glycerol, 0.5 g of maize extract (CSL; corn steep liquor), 1.2 g of ammonium nitrate, 0.3 g of hydrated magnesium sulfate, 0.3 g of magnesium oxide, 0.315 g of calcium hydroxide, 0.05 g of monopotassium phosphate and 0.380 g of hydrated copper nitrate in 1 L of water and the pH of the medium may be adjusted to a value on the order of 2.8 to 3 with a nitric acid solution. The fermentation may be terminated after the sugar has been exhausted and when the acidity of the medium is at a maximum and stable. Samples of musts may be taken near the end of the fermentation to evaluate the content of the fermentation broth. This method of diammonium itaconate/itaconic acid production from Aspergillus terreus Thom, anamorph strain NRRL 1960 having ATCC accession number 10020™ culture and similar methods for itaconic acid production are also described in U.S. Pat. No. 5,457,040, U.S. Pat. No. 3,873,425 and by Magnuson and Lasure eds., Advances in Fungal Biotechnology for Industry, Agriculture, and Medicine, Chapter 12: Organic Acid Production in Filamentous Fungi, pages 307-340; Kluwer Academic/Plenum Publishers (2004). the subject matter of which is incorporated herein by reference.

Fermentation broths containing diammonium citraconate/citraconic acid can be produced from fermentation broths containing itaconic acid as follows. A first fermentation broth containing about 5% (w/w) to about 20% (w/w) itaconic acid may be prepared as described above. If necessary the first fermentation broth may be concentrated or diluted to obtain these, or other desired, itaconic acid concentrations. Cells may be removed from the first fermentation broth by centrifugation, filtration or other means. The itaconic acid may then be placed in free acid form, if necessary, by treatment of the first fermentation broth with an acid. Typically, pH may be between 1.5 and 5 and most preferably may be between 2 and 2.5. The first fermentation broth may then optionally be concentrated at about 50° C. to about 100° C. or at a pressure of about 100 mm to about 500 mm Hg. Preferably, the first fermentation broth obtained has an itaconic acid concentration of from about 20% (w/w) to about 80% (w/w), or more preferably from about 30% (w/w) to about 50% (w/w), relative to the weight of the broth. A solvent that is sparingly soluble in water and which forms an azeotrope when added, such as pseudocumene or cyclohexanone, and a catalyst, such as pyridinium phosphate, may then be added to the first fermentation broth to dehydrate and isomerize the itaconic acid to citraconic anhydride. The resulting second fermentation broth comprising citraconic anhydride, solvent and possibly other fermentation broth components may then be contacted with an aqueous solution at an appropriate pH so that the citraconic anhydride may be hydrolyzed. In this manner, a third fermentation broth may be produced for the use in the production of citraconic acid.

For example, 2000 kg of an Aspergillus spp. mycelium-free, first fermentation broth having an itaconic acid concentration of 9.1% (w/w) relative to the weight of the broth may be obtained. Concentration to 108° C. may be performed to distill off, and remove, essentially all of the water from the first fermentation broth. An equal weight of pseudocumene solvent and 5 g of pyridinium phosphate catalyst may then be added to the first fermentation broth. Any remaining water may be distilled off at atmospheric pressure until no more comes off. The resulting second fermentation broth may be an organic solution containing 28.2 g of citraconic anhydride with a yield based on the itaconic acid content of the first fermentation broth of 18%. Alternatively, 3% (w/w) concentrated sulphuric acid relative to the weight of the itaconic acid in the first fermentation broth may be added to the first fermentation broth prior to concentration and the other steps described above are identically performed to produce an organic solution containing 119 g of citraconic anhydride with a yield of based on the itaconic acid content of the first fermentation broth of 76%. The production of second fermentation broths containing citraconic anhydride from a first fermentation broth produced by Aspergillus spp. culture, or the culture of another itaconic acid producing microorganism, is also described in U.S. Pat. No. 5,824,820 the subject matter of which is incorporated herein by reference. Such second fermentation broths comprising citraconic anhydride, solvent and possibly other fermentation broth components may then be contacted with an aqueous solution at an appropriate pH so that the citraconic anhydride may be hydrolyzed. In this manner, a third fermentation broth may be used for the production of citraconic acid.

Fermentation broths containing diammonium muconate/muconic acid may be produced from the Pseudomonas putida (Trevisan) Migula strain MW 1211.12 having ATCC accession number 31916™, or other such strains, by aerobic culture as follows. Inoculums of Pseudomonas putida (Trevisan) Migula strain MW 1211.12 having ATCC accession number 31916™ are prepared in 250 ml shake flasks containing 50 ml of a sterilized liquid medium designated NO comprising 20 mM of sodium succinate, 7.1 g/L of Na₂HPO₄, 13.6 g/L KH₂PO₄, 2.25 g/L (NH₄)₂SO₄, 0.246 g/L MgSO₄. 7H₂O, 0.0147 g/L of CaCl₂. 2H₂O, 0.00278 g/L of FeSO₄. 7H₂O and distilled water. The inoculum may be prepared by culture at 30° C. with shaking at 250 rpm for about 20 to about 24 hours to a turbidity of 200-240 klett units. A fed-batch fermentation may be conducted in a 16 L fermentor containing 12 L of a sterilized, liquid medium designated LP-1 having an NaOH adjusted pH of 6.9 and comprising 20 mM of sodium acetate, 0.426 g/L of Na₂HPO₄, 0.817 g/L KH₂PO₄, 1.12 g/L (NH₄)₂SO₄, 0.738 g/L MgSO₄. 7H₂O, 0.0294 g/L of CaCl₂. 2H₂O, 0.0167 g/L of FeSO₄.7H₂O, 3.9 g/L acetic acid and distilled water. The fermenter may be inoculated with 150 mL of inoculum and toluene may be supplied to the fermentation medium in vapor phase via air stripping at an air toluene vapor flow rate of 125 cc/min. The fermentation temperature may be maintained at 30° C., pH may be maintained at 6.9 with 10 M NH₄OH and 1 M H₂SO₄ solutions, dissolved oxygen may be maintained at 30-90% saturation with 600 RPM agitation and 5 liter/min aeration (or approximately 0.5 VVM). PLURONIC™ L61 polyol (BASF) may be used as an antifoam agent. As the turbidity of the fermentation medium reaches 90-110 klett units (about 9-15 hours after inoculation), an aqueous solution containing 10% (w/w) acetic acid, 0.114% (w/w) Na₂HPO₄ and 0.218% (w/w) KH₂PO₄ may be added to the fermentor medium at a rate of 0.4 mL/min. The air-toluene vapor rate may then be increased to 250 cc/min and increased again to 500 cc/min as the broth turbidity reaches 250 klett units. The air-toluene vapor rate may be eventually increased to 750 cc/min as the turbidity reaches 450-550 klett units and a muconic acid product concentration of 15 g/l is achieved in the fermentation broth. The fed-batch fermentation is normally continued for 24-36 h. The resulting fermentation broth may then be filtered to remove cells with a ROMICON® hollow tube “cross-flow” ultrafilter which has a polysulfone type ultrafiltration membrane (PM-100, molecular weight cutoff 100,000). The pH of the resulting clear, essentially cell-free fermentation broth may then be adjusted to pH 1-1.5 or another pH as desired, or necessary, with concentrated H₂SO₄.

A maximum specific productivity of 1.4 g/gdw/hr may be achieved during the fed-batch fermentation. The fermentation may be conducted by restricting the cell growth throughout the fed-batch fermentation cycle. During the early phase of fermentation (i.e., 6-12 hours after inoculation of cells), the growth carbon source (20 mM or 1.2 g/L acetate) and total phosphate (3 mM) may be in excess to initiate the growth of cells from 0.5-1.0 klett unit (after inoculation) to 50-100 klett units with 1-2 mM muconic acid concentration with utilization of the growth carbon source and the phosphate. At this point, growth carbon source as well as the required phosphate level are fed to the fermentor to provide additional growth and enzyme induction. The production of fermentation broths from Pseudomonas putida (Trevisan) Migula strain MW 1211.12 having ATCC accession number 31916™, or other such strains in the medium described above and other similar mediums, is also described in U.S. Pat. No. 4,535,059 the subject matter of which is incorporated herein by reference.

Fermentation broths containing diammonium muconate/muconic acid may be produced from the Candida maltosa Komagata et al., anamorph, deposited as Candida cloacae Komagata et al. anamorph strain AJ 4719 having ATCC accession number 20184™, or other such strains, by aerobic culture as follows. The Candida maltosa Komagata et al., anamorph, deposited as Candida cloacae Komagata et al. anamorph strain AJ 4719 having ATCC accession number 20184™ may be inoculated into a sterilized liquid medium comprising 1 g/L of catechol, 4 g/L of NH₄NO₃, 4 g/L NaCl, 1.15 g/L Na₂HPO₄, 0.2 g/L of KH₂PO₄, 0.1 g/L of KCl, 10 mg/L of MgSO₄. 7H₂O, 10 mg/L CaCl₂. 2H₂O), 5 mg/L of FeSO₄. 7H₂O and 500 mg/L of yeast extract in deionized water. Cultures may then be grown at 30° C. for several days and the fermentation broth may be centrifuged to remove cells. The pH of the resulting essentially, cell-free fermentation broth may then be adjusted to about pH 2.0 or another pH as desired, or necessary, with 2 N HCl. concentrated H₂SO₄. The production of fermentation broths from the Candida maltosa Komagata et al., anamorph, deposited as Candida cloacae Komagata et al. anamorph strain AJ 4719 having ATCC accession number 20184™, or other such strains, in the medium described above is also described in Gomi and Horiguchi, 52 Agric. Biol. Chem. 585 (1988) the subject matter of which is incorporated herein by reference.

Fermentation broths containing diammonium sebacate/sebacic acid may be produced as follows. Yeasts such as Candida spp., Torulopsis spp., Debaryomyces spp., Hansenula spp. and Pichia spp. may be cultured in sterile, mediums containing n-decane as the primary carbon source, such as a modified YM broth (ATCC medium 200) lacking dextrose or equivalents of other yeast culture mediums known in the art containing n-decane as the primary carbon source, under standard culture conditions to produce a fermentation broth. It is preferred that fermentations broths prepared by yeast culture be prepared from the culture of Candida tropicalis strains, such as Candida tropicalis (Castellani) Berkhout, anamorph strain OH23 having ATCC accession number 24887™, in modified YM broth (ATCC medium 200) medium containing n-decane as the primary carbon source and lacking dextrose. The production of fermentation broths from Candida tropicalis, or other such strains and yeast species, is also described in Ulezlo and Rogozhin, 40 Prikl Biokhim Mikrobiol. 533 (2004) the subject matter of which is incorporated herein by reference.

Fermentation broths containing diammonium sebacate/sebacic acid may be produced as follows. An inoculum containing one loopful of the yeast Torulopsis candida strain NC-3-58 derived from Torulopsis candida strain 99 grown on an slant of YM agar medium may be inoculated into 5 ml of a sterile, liquid medium designated decane medium that comprises 50 ml n-decane, 2 g NH₄H₂PO₄, 4 g K₂HPO₄, 0.5 g MgSO₄. 7H₂O, 1 g BACTO™ yeast extract, 1 mg MnSO₄4.nH₂O, 1 mg FeSO₄. 7H₂O and 1 mg ZnSO₄. 7H₂O in 950 ml of distilled water. Torulopsis candida strain NC-3-58 has a reduced bio-assimilation of sebacic acid relative to the Torulopsis candida strain 99 parent. The inoculum may then be cultured at 28° C. for 2 days on a reciprocal shaker at 200 rpm with a stroke amplitude of 2 cm. Then 5 ml of the cultured broth may be inoculated into 66.5 ml of decane medium in a 500 ml flask and cultured on the same shaker at 28° C. for 5-8 days. During the course of the fermentation, the pH of the medium may be kept at 7.5 by the addition of 2 N NaOH twice a day. A total of 1.5 L of inoculum broth should be prepared in this fashion. A sterilized, liquid medium having a pH of 7.0 and comprising 50 ml n-decane, 3 g NH₄H₂PO₄, 4 g K₂HPO₄, 0.2 g MgSO₄4.nH₂O, 1 g BACTO™ yeast extract, 1 g casamino acid, 1 mg MnSO₄4.nH₂O, 1 mg ZnSO₄. 7H₂O, 1 mg FeSO₄. 7H₂O and 10 μg biotin in 950 ml of distilled water may then be prepared. This medium may also be modified to contain corn steep liquor, malt extract, POLYPEPTON™, casamino acids, vitamin mixtures and combinations thereof A 30 L jar fermenter (Marubishi MSJS-30L) containing 13.5 L of this medium may then be inoculated with 1.5 L of the inoculum broth. Culture may then be performed at an agitation rate of 600 rpm, a temperature of 25° C. and a pH maintained at 6.5 for 83 h or longer (e.g., 6 days etc.) until the fermentation broth contains the desired concentration. Fermentation broths may thus be obtained using this process. The production of fermentation broths from Torulopsis candida strain NC-3-58 derived from Torulopsis candida strain 99, and other such strains, is also described in Kaneyuki et al., 58 J. Ferment. Technol. 405 (1980) the subject matter of which is incorporated herein by reference.

Fermentation broths containing diammonium sebacate/sebacic acid may be produced from the Candida tropicalis strain having FERM-P number 3291, or other such strains, by aerobic culture as follows. The FERM-P number is an accession number assigned by the Fermentation Research Institute, Agency of Industrial Science and Technology, at No. 5-2, 4-chome, Inagehigashi, Chiba-shi, Japan, from which the microorganisms having the FERM-P numbers are available. The tropicalis strain having FERM-P number 3291 and the ability to produce a long-chain dicarboxylic acid from a straight-chain hydrocarbon may be cultured in an incubator containing an appropriate liquid medium, such as YM broth, to obtain 120 L of inoculum containing 10-15 g/L of cultured fungal body, and the inoculum may be placed in a reactor supplied with 1200 L of an appropriate sterilized culture medium, such as a modified YM broth containing 240 L of n-decane (and optionally lacking dextrose). This medium may be mixed and adjusted to pH 5 and cultured at 32° C. for 12 h, during which time sterile air may be supplied at a rate of 400 L/min. Since the pH of the medium tends to drop during culture, a 10 N KOH solution may be added as necessary to maintain the pH of the medium at 5.0+/−0.1. After 12 h, the pH of the medium may be shifted to 7.0 and the culture may be further continued for an additional 72 h. After this a fermentation broth of 1200 L containing diammonium sebacate/sebacic acid and cultured fungal body may be obtained. A bleaching powder and/or hypochlorite may then be added to about 50 ppm to the fermentation broth, stirred, exposed to the atmosphere and left under this condition for 5 days to avoid after-fermentation as desired, or necessary. The pH of the fermentation broth can similarly be adjusted as desired, or necessary. The production of fermentation broths containing dicarboxylic acid from straight-chain, saturated hydrocarbons, such as n-decane, as a substrate with the Candida tropicalis strain having FERM-P number 3291, or other such strains, is also described in U.S. Pat. No. 4,339,536 the subject matter of which is incorporated herein by reference.

Fermentation broths containing diammonium dodecanedionate/dodecanedioic acid may be produced from the Candida tropicalis strain having FERM-P number 3291, or other such strains, by aerobic culture as follows. The tropicalis strain having FERM-P number 3291 and the ability to produce a long-chain dicarboxylic acid from a straight-chain hydrocarbon may be cultured in an incubator containing an appropriate liquid medium, such as YM broth, to obtain 120 L of inoculum containing 10-15 g/L of cultured fungal body, and the inoculum placed in a reactor supplied with 1200 L of an appropriate sterilized culture medium, such as a modified YM broth containing 240 L of n-dodecane (and optionally lacking dextrose). This medium may be mixed and adjusted to pH 5 and cultured at 32° C. for 12 h, during which time sterile air may be supplied at a rate of 400 L/min. Since the pH of the medium tends to drop during culture, a 10 N KOH solution may be added as necessary to maintain the pH of the medium at 5.0+/−0.1. After 12 h, the pH of the medium may be shifted to 7.0 and the culture may be further continued for an additional 72 h. After this a fermentation broth of 1200 L and a pH of 7.25 containing 42 kg/m³ of dodecanedioic acid (1,10-decamethylenedicarboxylic acid), 0.9 kg/m³ of dodecanoic acid, 11 kg/m³ of n-dodecanoic acid and 22 kg/m³ of cultured fungal body may be obtained. A bleaching powder and/or hypochlorite may then be added to 50 ppm to the fermentation broth, stirred, exposed to the atmosphere and left under this condition for 5 days to avoid after-fermentation as desired, or necessary. The pH may similarly be adjusted as desired, or necessary. The production of fermentation broths from the Candida tropicalis strain having FERM-P number 3291, or other such strains, is also described in U.S. Pat. No. 4,339,536 the subject matter of which is incorporated herein by reference.

A fermentable carbon source (e.g., carbohydrates and sugars), optionally a source of nitrogen and complex nutrients (e.g., corn steep liquor), additional media components such as vitamins, salts and other materials that can improve cellular growth and/or product formation, and water may be fed to the growth vessel 12 for growth and sustenance of the microbial culture. Typically, the microbial culture is grown under aerobic conditions provided by sparging an oxygen-rich gas (e.g., air or the like). Typically, an acid (e.g., sulphuric acid or the like) and ammonium hydroxide are provided for pH control during the growth of the microbial culture.

In one example (not shown), the aerobic conditions in growth vessel 12 (provided by sparging an oxygen-rich gas) are switched to anaerobic conditions by changing the oxygen-rich gas to an oxygen-deficient gas (e.g., CO₂ or the like). The anaerobic environment triggers bioconversion of the fermentable carbon source to HOOC—R—COOH compound acid in situ in growth vessel 12. Ammonium hydroxide is provided for pH control during bioconversion of the fermentable carbon source to HOOC—R—COOH compound acid. The HOOC—R—COOH compound acid that is produced is at least partially if not totally neutralized to NH₄⁺⁻OOC—R—COO⁻NH₄⁺ compound due to the presence of the ammonium hydroxide, leading to the production of a broth comprising NH₄⁺⁻OOC—R—COO⁻NH₄⁺ compound. The addition of CO₂ may provide an additional source of carbon for the production of HOOC—R—COOH compound acid.

In another example, the contents of growth vessel 12 may be transferred via stream 14 to a separate bioconversion vessel 16 for bioconversion of a carbohydrate source to HOOC—R—COOH compound acid. An oxygen-deficient gas (e.g., CO₂ or the like) is sparged in bioconversion vessel 16 to provide anaerobic conditions that trigger production of HOOC—R—COOH compound acid. Ammonium hydroxide is provided for pH control during bioconversion of the carbohydrate source to HOOC—R—COOH compound acid. Due to the presence of the ammonium hydroxide, the HOOC—R—COOH compound acid produced is at least partially neutralized to NH₄⁺⁻OOC—R—COO⁻NH₄⁺ compound, leading to production of a broth that comprises NH₄⁺⁻OOC—R—COO⁻NH₄⁺ compound. The addition of CO₂ may provide an additional source of carbon for production of HOOC—R—COOH compound acid.

In yet another example, the bioconversion may be conducted at relatively low pH (e.g., 3-6). A base (ammonium hydroxide or ammonia) may be provided for pH control during bioconversion of the carbohydrate source to HOOC—R—COOH compound acid. Depending of the desired pH, due to the presence or lack of the ammonium hydroxide, either HOOC—R—COOH compound acid is produced or the HOOC—R—COOH compound acid produced is at least partially neutralized to NH₄⁺⁻OOC—R—COOH, NH₄⁺⁻OOC—R—COO⁻NH₄⁺ compound or a mixture comprising HOOC—R—COOH compound acid, NH₄⁺⁻OOC—R—COOH and/or NH₄⁺⁻OOC—R—COO⁻NH₄⁺ compound. Thus, the HOOC—R—COOH compound acid produced during bioconversion can be subsequently neutralized, optionally in an additional step, by providing either ammonia or ammonium hydroxide leading to a broth comprising NH₄⁺⁻OOC—R—COO⁻NH₄⁺ compound. As a consequence, a “NH₄⁺⁻OOC—R—COO⁻NH₄⁺ compound-containing fermentation broth” generally means that the fermentation broth comprises NH₄⁺⁻OOC—R—COO⁻NH₄⁺ compound and possibly any number of other components such as NH₄⁺⁻OOC—R—COOH and/or HOOC—R—COOH compound acid, whether added and/or produced by bioconversion or otherwise. Similarly, a “NH₄⁺⁻OOC—R—COOH-containing fermentation broth” generally means that the fermentation broth comprises NH₄⁺³¹ OOC—R—COOH and possibly a number of other components such as NH₄⁺⁻OOC—R—COO⁻NH₄⁺ and/or HOOC—R—COOH, whether added and/or produced by bioconversion or otherwise.

The broth resulting from the bioconversion of the fermentable carbon source (in either growth vessel 12 or vessel 16, depending on where the bioconversion takes place), typically contains insoluble solids such as cellular biomass and other suspended material, which are transferred via stream 18 to clarification apparatus 20 before distillation. Removal of insoluble solids clarifies the broth. This reduces or prevents fouling of subsequent distillation equipment. The insoluble solids can be removed by any one of several solid-liquid separation techniques, alone or in combination, including but not limited to centrifugation and filtration (including, but not limited to ultra-filtration, micro-filtration or depth filtration). The choice of filtration can be made using techniques known in the art. Soluble inorganic compounds can be removed by any number of known methods such as, but not limited to, ion-exchange, physical adsorption and the like.

An example of centrifugation is a continuous disc stack centrifuge. It may be useful to add a polishing filtration step following centrifugation such as dead-end or cross-flow filtration, which may include the use of a filter aide such as diatomaceous earth or the like, or more preferably ultra-filtration or micro-filtration. The ultra-filtration or micro-filtration membrane can be ceramic or polymeric, for example. One example of a polymeric membrane is SelRO MPS-U20P (pH stable ultra-filtration membrane) manufactured by Koch Membrane Systems (850 Main Street, Wilmington, Mass., USA). This is a commercially available polyethersulfone membrane with a 25,000 Dalton molecular weight cut-off which typically operates at pressures of 0.35 to 1.38 MPa (maximum pressure of 1.55 MPa) and at temperatures up to 50° C. Alternatively, a filtration step may be employed alone using ultra-filtration or micro-filtration.

The resulting clarified NH₄⁺⁻OOC—R—COO⁻NH₄⁺ compound-containing broth or NH₄⁺⁻OOC—R—COOH compound-containing broth, substantially free of the microbial culture and other solids, is transferred via stream 22 to distillation apparatus 24.

Water and ammonia are removed from distillation apparatus 24 as an overhead, and at least a portion is optionally recycled via stream 26 to bioconversion vessel 16 (or growth vessel 12 operated in the anaerobic mode).

Under some conditions, distillation temperature and pressure are not critical as long as the distillation is carried out in a way that ensures that the distillation overhead contains water and ammonia, and the distillation bottoms comprises at least some NH₄⁺⁻OOC—R—COOH compound and at least about 20 wt % water. A more preferred amount of water is at least about 30 wt % and an even more preferred amount is at least about 40 wt %. The rate of ammonia removal from the distillation step increases with increasing temperature and also can be increased by injecting steam (not shown) during distillation. The rate of ammonia removal during distillation may also be increased by conducting distillation under a vacuum or by sparging the distillation apparatus with a non-reactive gas such as air, nitrogen or the like.

Removal of water during the distillation step can be enhanced by the use of an organic azeotroping agent such as toluene, xylene, methylcyclohexane, methyl isobutyl ketone, cyclohexane, heptane or the like, provided that the bottoms contains at least about 20 wt % water. If the distillation is carried out in the presence of an organic agent capable of forming an azeotrope consisting of the water and the agent, distillation produces a biphasic bottoms that comprises an aqueous phase and an organic phase, in which case the aqueous phase can be separated from the organic phase, and the aqueous phase used as the distillation bottoms. Byproducts such as amides and imides are substantially avoided provided the water level in the bottoms is maintained at a level of at least about 30 wt %.

A preferred temperature for the distillation step is in the range of about 50 to about 300° C., depending on the pressure. A more preferred temperature range is about 150 to about 240° C., depending on the pressure. A distillation temperature of about 170 to about 230° C. is preferred. “Distillation temperature” refers to the temperature of the bottoms (for batch distillations this may be the temperature at the time when the last desired amount of overhead is taken).

Adding a water miscible organic solvent or an ammonia separating solvent facilitates deammoniation over a variety of distillation temperatures and pressures as discussed above. Such solvents include aprotic, bipolar, oxygen-containing solvents that may be able to form passive hydrogen bonds. Examples include, but are not limited to, diglyme, triglyme, sulfoxides such as dimethylsulfoxide (DMSO), amides such as dimethylformamide (DMF) and dimethylacetamide, sulfones such as dimethylsulfone, gamma-butyrolactone (GBL), sulfolane, polyethyleneglycol (PEG), butoxytriglycol, N-methylpyrolidone (NMP), ethers such as dioxane, methyl ethyl ketone (MEK) and the like. Such solvents aid in the removal of ammonia from the NH₄⁺⁻OOC—R—COO⁻NH₄⁺ or NH₄⁺⁻OOC—R—COOH compound in the clarified broth. Regardless of the distillation technique, it is important that the distillation be carried out in a way that ensures that at least some NH₄⁺⁻OOC—R—COOH compound and at least about 20 wt % water remain in the bottoms and even more advantageously at least about 30 wt %. The distillation can be performed at atmospheric, sub-atmospheric or super-atmospheric pressures.

Under other conditions such as when the distillation is conducted in the absence of an azeotropic agent or ammonia separating solvent, the distillation is conducted at super atmospheric pressure at a temperature of greater than 100° C. to about 300° C. to form an overhead that comprises water and ammonia and a liquid bottoms that comprises HOOC—R—COOH compound acid and at least about 20 wt % water. Super atmospheric pressure typically falls within a range of greater than ambient atmosphere up to and including about 25 atmospheres. Advantageously the amount of water is at least about 30 wt %.

The distillation can be a one-stage flash, a multistage distillation (i.e., a multistage column distillation) or the like. The one-stage flash can be conducted in any type of flasher (e.g., a wiped film evaporator, thin film evaporator, thermosiphon flasher, forced circulation flasher and the like). The multistages of the distillation column can be achieved by using trays, packing or the like. The packing can be random packing (e.g., Raschig rings, Pall rings, Berl saddles and the like) or structured packing (e.g., Koch-Sulzer packing, Intalox packing, Mellapak and the like). The trays can be of any design (e.g., sieve trays, valve trays, bubble-cap trays and the like). The distillation can be performed with any number of theoretical stages.

If the distillation apparatus is a column, the configuration is not particularly critical, and the column can be designed using well known criteria. The column can be operated in either stripping mode, rectifying mode or fractionation mode. Distillation can be conducted in either batch, semi-continuous or continuous mode. In the continuous mode, the broth is fed continuously into the distillation apparatus, and the overhead and bottoms are continuously removed from the apparatus as they are formed. The distillate from distillation is an ammonia/water solution, and the distillation bottoms is a liquid, aqueous solution of NH₄⁺⁻OOC—R—COOH compound and HOOC—R—COOH compound acid, which may also contain other fermentation by-product salts (i.e., ammonium acetate, ammonium formate, ammonium lactate and the like) and color bodies.

The distillation bottoms can be transferred via stream 28 to cooling apparatus 30 and cooled by conventional techniques. Cooling technique is not critical. A heat exchanger (with heat recovery) can be used. A flash vaporization cooler can be used to cool the bottoms down to about 15° C. Cooling below 15° C. typically employs a refrigerated coolant such as, for example, glycol solution or, less preferably, brine. A concentration step can be included prior to cooling to help increase product yield. Further, both concentration and cooling can be combined using known methods such as vacuum evaporation and heat removal using integrated cooling jackets and/or external heat exchangers.

We found that the presence of some NH₄⁺⁻OOC—R—COOH in the liquid bottoms facilitates cooling-induced separation of the bottoms into a liquid portion in contact with a solid portion that at least “consists essentially” of HOOC—R—COOH compound acid (meaning that the solid portion is at least substantially pure crystalline HOOC—R—COOH compound acid) by reducing the solubility of HOOC—R—COOH compound acid in the liquid, aqueous, NH₄⁺⁻OOC—R—COOH-containing bottoms. The solubility of HOOC—R—COOH compound acid is reduced in aqueous NH₄⁺⁻OOC—R—COOH solutions. We discovered, therefore, that HOOC—R—COOH compound acid can be more completely crystallized out of an aqueous solution if some NH₄⁺⁻OOC—R—COOH is also present in that solution. This phenomenon allows crystallization of HOOC—R—COOH compound acid (i.e., formation of the solid portion of the distillation bottoms) at temperatures higher than those that would be required in the absence of NH₄⁺⁻OOC—R—COOH.

The distillation bottoms, after cooling, is fed via stream 32 to separator 34 for separation of the solid portion from the liquid portion. Separation can be accomplished via pressure filtration (e.g., using Nutsche or Rosenmond type pressure filters), centrifugation and the like. The resulting solid product can be recovered as product 36 and dried, if desired, by standard methods.

After separation, it may be desirable to treat the solid portion to ensure that no liquid portion remains on the surface(s) of the solid portion. One way to minimize the amount of liquid portion that remains on the surface of the solid portion is to wash the separated solid portion with water and dry the resulting washed solid portion (not shown). A convenient way to wash the solid portion is to use a so-called “basket centrifuge” (not shown). Suitable basket centrifuges are available from The Western States Machine Company (Hamilton, Ohio, USA).

The liquid portion of the separator 35 (i.e., the mother liquor) may contain remaining dissolved HOOC—R—COOH compound acid, any unconverted NH₄⁺⁻OOC—R—COOH, any fermentation byproducts such as ammonium acetate, lactate, or formate, and other minor impurities. This liquid portion can be fed via stream 38 to a downstream apparatus 40. In one instance, apparatus 40 may be a means for making a de-icer by treating in the mixture with an appropriate amount of potassium hydroxide, for example, to convert the ammonium salts to potassium salts. Ammonia generated in this reaction can be recovered for reuse in the bioconversion vessel 16 (or growth vessel 12 operating in the anaerobic mode). The resulting mixture of potassium salts is valuable as a de-icer and anti-icer.

The mother liquor from the solids separation step 34, can be recycled (or partially recycled) to distillation apparatus 24 via stream 42 to further enhance recovery of HOOC—R—COOH compound, as well as further convert NH₄⁺⁻OOC—R—COOH compound to HOOC—R—COOH compound acid.

The solid portion of the cooling-induced crystallization is substantially pure HOOC—R—COOH compound acid and is, therefore, useful for the known utilities of HOOC—R—COOH compound acid.

HPLC can be used to detect the presence of nitrogen-containing impurities such as amic acid, amide and imide of HOOC—R—COOH compound acid. The purity of HOOC—R—COOH compound acid can be determined by elemental carbon and nitrogen analysis. An ammonia electrode can be used to determine a crude approximation of HOOC—R—COOH compound acid purity.

Depending on the circumstances and various operating inputs, there are instances when the fermentation broth may be a clarified NH₄⁺⁻OOC—R—COOH-containing fermentation broth or a clarified HOOC—R—COOH compound acid-containing fermentation broth. In those circumstances, it can be advantageous to add NH₄⁺⁻OOC—R—COO⁻NH₄⁺, NH₄⁺⁻OOC—R—COOH and/or HOOC—R—COOH to those fermentation broths and, optionally, ammonia and/or ammonium hydroxide to facilitate the production of substantially pure HOOC—R—COOH compound acid. For example, the operating pH of the fermentation broth may be oriented such that the broth is a NH₄⁺⁻OOC—R—COOH compound-containing broth or a HOOC—R—COOH compound acid-containing broth. NH₄⁺⁻OOC—R—COOH, NH₄⁺⁻OOC—R—COO⁻NH₄⁺, HOOC—R—COOH, ammonia and/or ammonium hydroxide may optionally be added to those broths to attain a broth pH less than about 6 to facilitate production of the above-mentioned substantially pure HOOC—R—COOH compound acid. In one particular form, it is especially advantageous to recycle HOOC—R—COOH, NH₄⁺⁻OOC—R—COOH and water from the liquid bottoms resulting from the distillation step 24 and/or the liquid portion from the separator 34 into the fermentation broth and/or clarified fermentation broth. In referring to the NH₄⁺⁻OOC—R—COOH-containing broth, such broth generally means that the fermentation broth comprises NH₄⁺⁻OOC—R—COOH and possibly any number of other components such as NH₄⁺⁻OOC—R—COO⁻NH₄⁺ compound and/or HOOC—R—COOH compound acid, whether added and/or produced by bioconversion or otherwise.

A synthetic, aqueous NH₄⁺⁻OOC—R—COO⁻NH₄⁺ compound solution may be used in place of an actual clarified NH₄⁺⁻OOC—R—COO⁻NH₄⁺ compound-containing fermentation broth.

The use of a synthetic NH₄⁺⁻OOC—R—COO⁻NH₄⁺ compound solution is believed to be a good model for the behavior of an actual broth in our processes because of the solubility and acidity of the typical fermentation by-products found in actual broth. Typically, the major by-products produced during fermentation are salts of monocarboxylic acids such as ammonium lactate and ammonium formate. If these impurities are present during the distillation step, one would not expect them to lose ammonia and form free acids in significant quantities until all of the NH₄⁺⁻OOC—R—COO⁻NH₄⁺ compound had been converted to HOOC—R—COOH compound acid. This is because acetic acid, lactic acid and formic acid tend to be stronger acids than the second acid group of HOOC—R—COOH compound acids such as malonic acid (pKa=5.69), malic acid (pKa=5.13), citraconic acid (pKa=6.15 to 6.2), itaconic acid (pKa=5.45), muconic acid, sebacid acid (pKa=5.450), and dodecandioic. In other words, lactate, formate and even monohydrogen fumarate are weaker bases than the dianion fumarate. Furthermore, ammonium lactate and ammonium formate tend to be significantly more soluble in water than HOOC—R—COOH compound acid, and each is typically present in the broth at less than 10% of the NH₄⁺⁻OOC—R—COO⁻NH₄⁺ compound concentration. In addition, even if the acids (formic and lactic acids) were formed during the distillation step, they are miscible with water and tend not to crystallize from water. This means that the HOOC—R—COOH compound acid reaches saturation and crystallizes from solution (i.e., forming the solid portion), leaving the acid impurities dissolved in the mother liquor (i.e., the liquid portion).

### Example 1

This example demonstrates ammonia evolution from aqueous NH₄⁺⁻OOC—R—COO⁻ NH₄⁺ compounds such as diammonium malate, diammonium itaconate, diammonium malonate and diammonium dodecanedionate.

The outer necks of a three neck 1-L round bottom flask were fitted with a thermometer and a stopper. The center neck was fitted with a five tray 1″ Oldershaw section. The Oldershaw section was topped with a distillation head. An ice cooled 500 mL round bottom flask was used as the receiver for the distillation head. The 1-L round bottom flask was charged with distilled water, the HOOC—R—COOH compound acid indicated in Table 1 and concentrated ammonium hydroxide solution. The contents were stirred with a magnetic stirrer to dissolve all the solids. After the solids dissolved, the contents were heated with the heating mantle to distill 350 g of distillate. The distillate was collected in the ice cooled 500 mL round bottom flask. The pot temperature was recorded as the last drop of distillate was collected. The pot contents were allowed to cool to room temperature and the weight of the residue and weight of the distillate were recorded. The ammonia content of the distillate was then determined via titration. The results were recorded in Table 1.

TABLE 1

| Run # | 1 | 2 | 3 | 4 | 5 |
|---|---|---|---|---|---|
| Name of Acid | Malic | Itaconic | Malonic | DDA* | Citric |
| Wt Acid Charged (g) | 13.4 | 13.01 | 10.44 | 23.04 | 19.22 |
| Moles Acid Charged | 0.1 | 0.1 | 0.1 | 0.1 | 0.1 |
| Wt 28% NH₃ Solution | 12.11 | 12.1 | 12.18 | 12.1 | 18.27 |
| Charged (g) | | | | | |
| Moles NH₃ Charged | 0.2 | 0.2 | 0.2 | 0.2 | 0.3 |
| Wt Water Charged (g) | 800.21 | 800.54 | 800.1 | 800.1 | 800.24 |
| Wt Distillate (g) | 350.78 | 350.14 | 350.5 | 350 | 350.85 |
| Wt Residue (g) | 461.99 | 467 | 467 | 465 | 478.93 |
| % Mass Accountability | 98.5 | 99 | 99.4 | 97.6 | 99 |
| Wt % NH₃ in distillate | 0.07 | 0.11 | 0.29 | 0.13 | 0.2 |
| (titration) | | | | | |
| Moles NH₃ in distillate | 0.015 | 0.023 | 0.06 | 0.027 | 0.041 |
| % Total NH₃ removed in | 7.2 | 11.3 | 30 | 13 | 13.7 |
| Distillate | | | | | |
| % First NH₃ removed in | 14.4 | 22.6 | 60 | 26 | 41.1 |
| Distillate | | | | | |
| DiNH₄/MonoNH₄ | 86/14 | 77/23 | 40/60 | 74/26 | |
| Final Pot Temp (° C.) | 100 | 100 | 100 | 100 | 100 |
| Micromoles of NH₃/g | 41 | 65 | 171 | 77 | 117 |
| distillate | | | | | |
| Initial Wt % ammonium | 2 | 2.1 | 1.7 | 3.2 | 3 |
| salt | | | | | |
| pKa₁ | 3.4 | 3.85 | 2.83 | unk | 3.14 |
| pKa₂ | 5.11 | 5.45 | 5.69 | unk | 4.77 |
| pKa₃ | NA | NA | NA | NA | 6.39 |

Although our processes have been described in connection with specific steps and forms thereof, it will be appreciated that a wide variety of equivalents may be substituted for the specified elements and steps described herein without departing from the spirit and scope of this disclosure as described in the appended claims.

1. A process for making HOOC—R—COOH from a clarified NH₄⁺⁻OOC—R—COO⁻NH₄⁺-containing fermentation broth comprising:
(a) distilling the broth under super atmospheric pressure at a temperature of >100° C. to about 300° C. to form an overhead that comprises water and ammonia, and a liquid bottoms that comprises HOOC—R—COOH, and at least about 20 wt % water;
(b) cooling and/or evaporating the bottoms to attain a temperature and composition sufficient to cause the bottoms to separate into a liquid portion and a solid portion that is substantially pure HOOC—R—COOH; and
(c) separating the solid portion from the liquid portion.

2. The process of claim 1, wherein the liquid bottoms contains at least some NH₄⁺⁻OOC—R—COOH and/or the solid portion is substantially free of auric acid, amide and imide of HOOC—R—COOH compound acid.

3. The process of claim 1, further comprising recycling the HOOC—R—COOH and water and, optionally, NH₄⁺⁻OOC—R—COOH from the liquid bottoms to the clarified broth.

4. The process of claim 1, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

5. The process of claim 1, further comprising removing water from the liquid bottoms to increase concentration of HOOC—R—COOH in the liquid bottoms.

6. A process for making HOOC—R—COOH from a clarified NH₄⁺⁻OOC—R—COO⁻NH₄⁺-containing fermentation broth comprising:
(a) adding an ammonia separating and/or water azeotroping solvent to the broth;
(b) distilling the broth at a temperature and pressure sufficient to form an overhead that comprises water and ammonia, and a liquid bottoms that comprises HOOC—R—COOH, and at least about 20 wt % water;
(c) cooling and/or evaporating the bottoms to attain a temperature and composition sufficient to cause the bottoms to separate into a liquid portion and a solid portion that is substantially pure HOOC—R—COOH; and
(d) separating the solid portion from the liquid portion.

7. The process of claim 6, wherein the liquid bottoms contains at least some NH₄⁺⁻OOC—R—COOH and/or the solid portion is substantially free of amic acid, amide and imide of HOOC—R—COOH compound acid.

8. The process of claim 6, further comprising recycling the HOOC—R—COOH, water, and, optionally, the solvent and/or NH₄⁺⁻OOC—R—COOH from the liquid bottoms to the clarified broth.

9. The process of claim 6, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

10. The process of claim 6, further comprising removing water from the liquid bottoms to increase concentration of HOOC—R—COOH in the liquid bottoms.

11. A process for making HOOC—R—COOH from a clarified NH₄⁺⁻OOC—R—COOH-containing fermentation broth comprising:
(a) distilling the broth under super atmospheric pressure at a temperature of >100° C. to about 300° C. to form an overhead that comprises water and ammonia, and a liquid bottoms that comprises HOOC—R—COOH, and at least about 20 wt % water;
(b) cooling and/or evaporating the bottoms to attain a temperature and composition sufficient to cause the bottoms to separate into a liquid portion and a solid portion that is substantially pure HOOC—R—COOH; and
(c) separating the solid portion from the liquid portion.

12. The process of claim 11, wherein the liquid bottoms contains at least some NH₄⁺⁻OOC—R—COOH and/or the solid portion is substantially free of auric acid, amide and imide of HOOC—R—COOH compound acid.

13. The process of claim 11, further comprising recycling the HOOC—R—COOH and water and optionally NH₄⁺⁻OOC—R—COOH from the liquid bottoms to the clarified broth.

14. The process of claim 11, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

15. The process of claim 11, further comprising removing water from the liquid bottoms to increase concentration of HOOC—R—COOH in the liquid bottoms.

16. A process for making HOOC—R—COOH from a clarified NH₄⁺⁻OOC—R—COOH⁻ containing fermentation broth comprising:
(a) adding an ammonia separating and/or water azeotroping solvent to the broth;
(b) distilling the broth at a temperature and pressure sufficient to form an overhead that comprises water and ammonia, and a liquid bottoms that comprises HOOC—R—COOH, and at least about 20 wt % water;
(c) cooling and/or evaporating the bottoms to attain a temperature and composition sufficient to cause the bottoms to separate into a liquid portion and a solid portion that is substantially pure HOOC—R—COOH; and
(d) separating the solid portion from the liquid portion.

17. The process of claim 16, wherein the liquid bottoms contains at least some NH₄⁺⁻OOC—R—COOH and/or the solid portion is substantially free of auric acid, amide and imide of HOOC—R—COOH compound acid.

18. The process of claim 16, further comprising recycling the HOOC—R—COOH, water, and, optionally, the solvent and/or NH₄⁺⁻OOC—R—COOH from the liquid bottoms to the clarified broth.

19. The process of claim 16, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

20. The process of claim 16, further comprising removing water from the liquid bottoms to increase concentration of HOOC—R—COOH in the liquid bottoms.

21. The process of claim 1, wherein the fermentation broth is obtained by fermenting a carbon source in the presence of a microorganism selected from the group consisting of the Phanerochaete chrysorporium strain having ATCC accession number 24725™; Corynebacterium nitrilophilus strain having ATCC accession number 21419™; Gordona terrae strain having accession number FERM-BP-4535; Rhodococcus rhodochrous strain having ATCC accession number 33025™; Phanerochaete chrysorporium strain having ATCC accession number 24725™; Gordona terrae strain having accession number FERM-BP-4535; Corynebacterium nitrilophilus strain having ATCC accession number 21419™; Rhodococcus rhodochrous strain having ATCC accession number 33025™; Aspergillus flavus strain having ATCC accession number 13697™; Aspergillus flavus strain having ATCC accession number 13698™; Aspergillus parasiticus strain having ATCC accession number 16869™; Aspergillus parasiticus strain having ATCC accession number 13696™; Aspergillus oryzae strain having ATCC accession number 56747™; Candida tropicalis strain having ATCC accession number 24887™; Rhodotorula mucilanginosa HOOC—R—COOH strain having ATCC accession number 64041™; lysine-requiring HOOC—R—COOH Saccharomyces cerevisiae strain C-1; Aspergillus terreus strain having ATCC accession number 10020™; Aspergillus terreus strain RC4′; Aspergillus terreus strain CM85J; Aspergillus terreus strain having ATCC accession number 10029™; Aspergillus terreus strain having ATCC accession number 20542™; Aspergillus terreus strain having ATCC accession number 32359™; Aspergillus terreus strain having ATCC accession number 32587™; Aspergillus terreus strain having ATCC accession number 32588™; Aspergillus terreus strain having ATCC accession number 32589™; Aspergillus terreus strain K having ATCC accession number 32590™; Aspergillus terreus strain 3 having ATCC accession number 36364™; Aspergillus terreus TN484-M1; Aspergillus itaconicus strain having ATCC accession number 56806™; Pseudomonas putida strain having ATCC accession number 31916™; Candida maltosa HOOC—R—COOH strain having ATCC accession number 20184™; Torulopsis candida strain NC-3-58 and Candida tropicalis strain having FERM-P number 3291.
